(19) United States
(12) Patent Application Publication
(10) Pub. No.: US 2011/0016546 A1
(43) Pub. Date: Jan. 20, 2011

(54) PORCINE GENOME EDITING WITH ZINC FINGER NUCLEASES

(75) Inventors: Joseph Bedell, St. Louis, MO (US); Brian Buntaine, St. Louis, MO (US); Xiaoxia Cui, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,893

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A01K 67/027; C12N 5/10

(52) U.S. Cl. 800/17; 435/325

(57) ABSTRACT

The present invention provides a genetically modified porcine or cell comprising at least one edited chromosomal sequence. In particular, the chromosomal sequence is edited using a zinc finger nuclease-mediated editing process. The disclosure also provides zinc finger nucleases that target specific chromosomal sequences in the porcine genome.

### CROSS-REFERENCE TO RELATED APPLICATIONS

This application claims the priority of U.S. provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified porcine or porcine cells comprising at least one edited chromosomal sequence. In particular, the invention relates to the use of targeted zinc finger nucleases to edit chromosomal sequences in the porcine.

### BACKGROUND OF THE INVENTION

The pork industry is a vital economic component of our economy, which produces many essential and varied products including meat, and concentrated protein products derived from pork.

Many phenotypic traits associated with pork production have been identified. The genetics of these phenotypes are well documented, but in many cases the actual genes that are responsible are yet to be characterized. The identification of genes controlling several traits of interest in pigs has been accomplished by positional candidate cloning. Once the location of a trait is determined by linkage to the markers, possible candidate genes controlling the trait can be inferred because of their proximity to linked markers. Subsets of genes that are mapped in humans and mice have also been mapped in pigs through comparative genomic study. Porcine genome mapping has been done by the NAGRP Pig Genome Coordination Program and the U.S. Pig Genome Map can be found at http://www.animalgenome.org/pigs/. Other informational databases on the genetic maps of pigs have also been done.

In addition to pork production, traits such as disease resistance and more environmentally sound breeding are also important for the pig industry. There is a need, therefore, for improved methods of knocking out genes coding undesirable proteins in pigs, as well as means of modifying genes involved in desirable phenotypes for higher economic value.

### SUMMARY OF THE INVENTION

One aspect of the present disclosure encompasses a genetically modified porcine comprising at least one edited chromosomal sequence.

A further aspect provides an porcine embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

Another aspect provides a genetically modified porcine cell comprising at least one edited chromosomal sequence.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a protein associated with porcine or human related disease or traits. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the protein associated with porcine or human related disease or traits generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with porcine or human related disease or traits using targeted zinc finger nuclease technology is rapid, precise, and highly efficient.

### (I) Genetically Modified Porcine

One aspect of the present disclosure provides a genetically modified porcine in which at least one chromosomal sequence encoding a disease- or trait-related protein has been edited. For example, the edited chromosomal sequence may be inactivated such that the sequence is not transcribed and/or a functional disease- or trait-related protein is not produced. Alternatively, the edited chromosomal sequence may be modified such that it codes for an altered disease- or trait-related protein. For example, the chromosomal sequence may be modified such that at least one nucleotide is changed and the expressed disease- or trait-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed disease- or trait-related protein comprises a single amino acid deletion or insertion, provided such a protein is functional. For example, a protein coding sequence may be inactivated such that the protein is not produced. Alternatively, a microRNA coding sequence may be inactivated such that the microRNA is not produced. Furthermore, a control sequence may be inactivated such that it no longer functions as a control sequence. The modified protein may have altered substrate specificity, altered enzyme activity, altered kinetic rates, and so forth. Furthermore, the edited chromosomal sequence may comprise an integrated sequence and/or a sequence encoding an orthologous protein associated with a disease or a trait. The genetically modified porcine disclosed herein may be heterozygous for the edited chromosomal sequence encoding a protein associated with a disease or a trait. Alternatively, the genetically modified porcine may be homozygous for the edited chromosomal sequence encoding a protein associated with a disease or a trait.

In one embodiment, the genetically modified porcine may comprise at least one inactivated chromosomal sequence encoding a disease- or trait-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional disease- or trait-related protein is not produced. The inactivated chromosomal sequence comprises no exogenously introduced sequence. Such a porcine may be termed a “knockout.” Also included herein are genetically modified porcines in which two, three, four, five, six, seven, eight, nine, or ten or more chromosomal sequences encoding proteins associated with a disease or a trait are inactivated.

In another embodiment, the genetically modified porcine may comprise at least one edited chromosomal sequence encoding an orthologous protein associated with a disease. The edited chromosomal sequence encoding an orthologous disease- or trait-related protein may be modified such that it codes for an altered protein. For example, the edited chromosomal sequence encoding a disease- or trait-related protein may comprise at least one modification such that an altered version of the protein is produced. In some embodiments, the edited chromosomal sequence comprises at least one modification such that the altered version of the disease-related protein results in the disease in the porcine. In other embodiments, the edited chromosomal sequence encoding a disease- or trait-related protein comprises at least one modification such that the altered version of the protein protects against a disease or does not form a trait in the porcine. The modification may be a missense mutation in which substitution of one nucleotide for another nucleotide changes the identity of the coded amino acid.

In yet another embodiment, the genetically modified porcine may comprise at least one chromosomally integrated sequence. The chromosomally integrated sequence may encode an orthologous disease- or trait-related protein, an endogenous disease- or trait-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a porcine or human disease- or trait-related protein may be integrated into a “safe harbor” locus, such as the Rosa26 locus, HPRT locus, or AAV locus. In one iteration of the disclosure an animal comprising a chromosomally integrated sequence encoding disease- or trait-related protein may be called a “knock-in”, and it should be understood that in such an iteration of the animal, no selectable marker is present. An animal comprising a chromosomally integrated sequence encoding a porcine or human disease- or trait-related protein may be called a “knock-in.” The present disclosure also encompasses genetically modified animals in which two, three, four, five, six, seven, eight, nine, or ten or more sequences encoding protein(s) associated with a disease or a trait are integrated into the genome.

In an exemplary embodiment, the genetically modified porcine may be a “humanized” porcine comprising at least one chromosomally integrated sequence encoding a functional human disease or trait-related protein. The functional human disease or trait-related protein may have no corresponding ortholog in the genetically modified porcine. Alternatively, the wild-type porcine from which the genetically modified porcine is derived may comprise an ortholog corresponding to the functional human disease or trait-related protein. In this case, the orthologous sequence in the “humanized” porcine is inactivated such that no functional protein is made and the “humanized” porcine comprises at least one chromosomally integrated sequence encoding the human disease or trait-related protein. Those of skill in the art appreciate that “humanized” porcines may be generated by crossing a knock out porcine with a knock in porcine comprising the chromosomally integrated sequence.

The chromosomally integrated sequence encoding a disease or trait-related protein may encode the wild type form of the protein. Alternatively, the chromosomally integrated sequence encoding a disease- or trait-related protein may comprise at least one modification such that an altered version of the protein is produced. In some embodiments, the chromosomally integrated sequence encoding a disease or trait-related protein comprises at least one modification such that the altered version of the protein produced causes a disease or forms a trait. In other embodiments, the chromosomally integrated sequence encoding a disease- or trait-related protein comprises at least one modification such that the altered version of the protein protects against the development of a disease or an undesirable trait.

In yet another embodiment, the genetically modified porcine may comprise at least one edited chromosomal sequence encoding a disease or trait-related protein such that the expression pattern of the protein is altered. For example, regulatory regions controlling the expression of the protein, such as a promoter or transcription binding site, may be altered such that the disease or trait-related protein is over-produced, or the tissue-specific or temporal expression of the protein is altered, or a combination thereof. Alternatively, the expression pattern of the disease or trait-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyse the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a disease or trait-related protein. The genetically modified porcine comprising the lox-flanked chromosomal sequence encoding a disease or trait-related protein may then be crossed with another genetically modified porcine expressing Cre recombinase. Progeny comprising the lox-flanked chromosomal sequence and the Cre recombinase are then produced, and the lox-flanked chromosomal sequence encoding a disease or trait-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a disease or trait-related protein.

In one exemplary example, Myostatin (GDF-8) is a member of the TGF-b superfamily of growth factors which is expressed predominantly in skeletal muscle. Myostatin is a purported negative regulator of muscle development. Myostatin knock-out mice (mice which have the myostatin gene specifically inactivated) have individual muscles which can weigh 2 to 3 times more than the same muscles in wild-type control mice. Furthermore, the double-muscled body type of Belgian Blue and Piedmontese cattle has been linked to an inactive myostatin gene. The loss of myostatin activity causes these cattle to be extremely muscular and lean. Porcine studies have shown that runt piglets have increased myostatin expression when compared to the more heavily muscled control piglets.

The production of animals with superior muscle structure is of great importance to food animal agriculture. Skeletal muscle is the major component of lean tissue that is used for food. In swine, there are observable differences in the carcass muscularity of different breeds of pigs. For example, Yorkshire pigs have shown a greater ability to synthesize proteins within muscle as well as having greater muscle weights than feral pigs of equal weight or age. Chinese Meishan pigs, on the other hand, have been shown to have poor growth rates and efficiency and fatty carcasses when compared to the occidental breeds.

Due to its role in muscle development in other mammals, it is important to investigate myostatin's role in swine muscle development. In swine, sequence variations in the GDF-8 coding region may lead to varying levels of myostatin expression among breeds and subsequently determine variation in muscle development. These same sequence differences may potentially serve as markers for muscle traits in swine. Producers often use genetic markers for herd and production management. GDF-8 may represent an ideal marker for muscle mass because it is a single gene, the absence of which has shown to increase muscle mass in both mice and cattle.

Exemplary examples of porcine chromosomal sequences to be deleted or edited in porcine include those that code for proteins such as Myostatin/GDF8 for increased muscle growth. In one embodiment, the genetically modified porcine may comprise an edited chromosomal sequence encoding Myostatin/GDF8 protein, wherein the edited chromosomal sequence comprises a mutation such that Myostatin/GDF8 is not produced. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the nonsense, deletion, or insertion mutation “inactivates” the sequence such that Myostatin/GDF8 protein is not produced. Thus, a genetically modified porcine comprising an inactivated Myostatin/GDF8 chromosomal sequence may produce increased muscle growth in pigs.

Exemplary examples of porcine chromosomal sequences to be deleted or edited in porcine include those that code for proteins that affect the coat color. The melanocortin receptor 1 (MC1R) plays a central role in regulation of eumelanin (black/brown) and phaeomelanin (red/yellow) synthesis within the mammalian melanocyte and is encoded by the classical Extension (E) coat color locus. Sequence analysis of MC1R from seven porcine breeds revealed a total of four allelic variants corresponding to five different E alleles. The European wild boar possessed a unique MC1R allele that is required for the expression of a wild-type coat color. Two different MC1R alleles were associated with the dominant black color in pigs. MC1R*2 was found in European Large Black and Chinese Meishan pigs and exhibited two missense mutations compared with the wild-type sequence. One of these mutations, L99P, may form a constitutively active receptor. MC1R*3 was associated with the black color in the Hampshire breed and involved a single missense mutation D121N. This same MC1R variant was also associated with E^{P}, one of the E alleles, which results in black spots on a white or red background. The genetically modified porcine comprising the edited chromosomal sequence described above will present different coat color and pattern than a porcine in which the chromosomal region is not edited.

Monocytes-macrophages, the target cells of African swine fever virus (ASFV) are highly heterogeneous in phenotype and function. Studies have shown the correlation between the phenotype of specific populations of porcine macrophages and their permissiveness to ASFV infection. Bone marrow cells and fresh blood monocytes were less susceptible to in vitro infection by ASFV than more mature cells, such as alveolar macrophages. FACS analyses of monocytes using a panel of mAbs specific for porcine monocyte/macrophages showed that infected cells had a more mature phenotype, expressing higher levels of several macrophage specific markers and SLA II antigens. Maturation of monocytes led to an increase in the percentage of infected cells, which correlated with an enhanced expression of CD163. Separation of CD163⁺ and CD163⁻ monocytes demonstrated the specific sensitivity of the CD163⁺ subset to ASFV infection. In vivo experiments also showed a close correlation between CD163 expression and virus infection. Altogether, these results strongly suggest a role of CD163 in the process of infection of porcine monocytes/macrophages by ASFV.

Additionally, porcine reproductive and respiratory syndrome virus (PRRSV) shows a very restricted tropism for cells of the monocyte/macrophage lineage. It enters cells via receptor-mediated endocytosis. A monoclonal antibody (MAb) that is able to block PRRSV infection of porcine alveolar macrophages (PAM) and that recognizes a 210-kDa protein (p210) has also been described (MAb41D3).

In one study, the p210 protein was purified from PAM by immunoaffinity using MAb41D3 and was subjected to internal peptide sequencing after tryptic digestion. Amino acid sequence identities ranging from 56 to 91% with mouse sialoadhesin, a macrophage-restricted receptor, were obtained with four p210 peptides. Using these peptide data, the full p210 cDNA sequence (5,193 bp) was subsequently determined. It shared 69 and 78% amino acid identity, respectively, with mouse and human sialoadhesins. Swine (PK-15) cells resistant to viral entry were transfected with the cloned p210 cDNA and inoculated with European or American PRRSV strains. Internalized virus particles were detected only in PK-15 cells expressing the recombinant sialoadhesin, demonstrating that this glycoprotein mediated uptake of both types of strains. Virus uncoating after fusion of the virus with the endocytic vesicle membrane, was also observed.

The ability of porcine sialoadhesin to mediate endocytosis has been demonstrated by specific internalization of MAb41D3 into PAM. Altogether, these results show that sialoadhesin is involved in the entry process of PRRSV in PAM and disease resistance can be obtained through PRRSV coat protein uncoating.

Further, scavenger receptor CD163 is a key entry mediator for PRRSV. In one study, CD163 protein domains involved in PRRSV infection were identified through the creation of deletion mutants and chimeric mutants. Infection experiments revealed that scavenger receptor cysteine-rich (SRCR) domain 5 (SRCR 5) is essential for PRRSV infection, while the four N-terminal SRCR domains and the cytoplasmic tail are not required. The remaining CD163 protein domains need to be present but can be replaced by corresponding SRCR domains from CD163-L1, resulting in reduced (SRCR 6 and interdomain regions) or unchanged (SRCR 7 to SRCR 9) infection efficiency. In addition, CD163-specific antibodies recognizing SRCR 5 are able to reduce PRRSV infection.

CD163 and sialoadhesin for disease resistance to ASFV and PSSRV are also exemplary examples of porcine chromosomal sequences to be deleted or edited in porcine include those that code for those proteins. In one embodiment, the genetically modified porcine may comprise an edited chromosomal sequence encoding the CD163 or sialoadhesin protein, wherein the edited chromosomal sequence comprises a mutation such that CD163 or sailoadhesin protein is not produced. The mutation may be a nonsense mutation in which substitution of one nucleotide for another introduces a stop codon, a deletion mutation in which one or more nucleotides are deleted from the chromosomal sequence, or an insertion mutation in which one or more nucleotides are introduced into the chromosomal sequence. Accordingly, the nonsense, deletion, or insertion mutation “inactivates” the sequence such that CD163 or sialoadhesion protein is not produced. Thus, a genetically modified porcine comprising an inactivated CD163 or sialoadhesion chromosomal sequence may produce increased disease resistance in pigs.

In another non-limiting embodiment, the genetically modified porcine may comprise an edited chromosomal sequence inactivating CD163 or sialoadhesion only in the forms of variants that are known to be generally susceptible to PRRSV or ASFV in certain porcine breeds.

Increased use of extremely lean, heavily muscled genotypes for terminal market porcine production has resulted in increased concern about quality problems in pork products. These problems focus primarily on color, firmness, water holding capacity and marbling in pork muscle. Pale, soft, exudative (PSE) pork is a general term used to describe these pork quality problems. It has been documented that the U.S. pork supply contains approximately 16% PSE pork. One genetic cause of PSE pork is associated with the presence of the halothane (HAL) gene, so named because pigs that carried two copies of the gene (called homozygous carriers or nn) were discovered to undergo physiological stress and die when exposed to halothane anesthesia. However, it has been shown that pigs carrying one copy of the HAL gene (called heterozygous carriers or Nn) tend to produce leaner carcasses but more PSE pork than HAL gene free pigs (called homozygous negative or NN).

The “acid meat” condition is very similar in characteristics to the pale, soft and exudative (PSE) pork condition caused by the PSS gene. In fact, when the PSS gene is present, it intensifies the effect of the RN gene on meat quality. Extensive research on the effects of the RN gene on pork production, especially on pork quality, has been carried out worldwide since it was discovered. Studies on the impact of the RN gene on fresh and processed pork quality have consistently shown that the RN gene has negative effects on meat pH, WHC, color, drip loss, cooking loss and processing yield.

The negative effects of the RN gene are a result of a dramatic increase in glycogen levels in the muscle of live pigs that have the gene. Glycogen is the form in which sugars are stored, particularly in liver and muscle. After slaughter, muscle glycogen is converted to lactic acid, which lowers the muscle pH. Therefore, the more glycogen the muscle contains, the more lactic acid will be produced and the lower the ultimate pH of the muscle will be. The increased lactic acid levels may result in muscle pH dropping below 5.5 within 24 hours after slaughter. This low and dramatic drop in meat pH causes a breakdown in protein, which results in pale muscle colour and poor WHC in the meat.

Therefore, in another embodiment, the genetically modified porcine may comprise an edited chromosomal sequence encoding HAL, RN, or PSS wherein the edited chromosomal sequence comprises at least one modification such that an altered version of HAL, RN or PSS is produced. Those of skill in the art will appreciate that many different modifications are possible in the HAL, RN and PSS coding regions. In one embodiment, the genetically modified porcine comprising a modified HAL chromosomal region for stress susceptibility. In other embodiments, the genetically modified porcine comprising a modified HAL, RN or PSS chromosomal region may have more muscle and less fat than a porcine in which the HAL, RN or PSS chromosomal region is not modified.

Litter size is one of the most important economic traits in pig production, and the more piglet numbers per litter is capable to increase pork production and bring more economic profit for pig industry. ESR (estrogen receptor) gene has been determined to be one of the major genes affecting phenotype of litter size without any genetic negative correlation to growth and carcass traits. In one study, an optimized standard PCR-RFLP protocol is employed to type 262 sows from 5 different breeds in ESR loci, and then with the computation based on linear model ESR gene is confirmed to be a major locus significantly associated with litter size. The genetic effect of ESR gene is quite large in these breeds, especially in these Chinese pig population. The sows of beneficial homozygote BB produce 1.40-3.37 total number born/litter and 0.63-3.58 number born alive/litter more than the sows of non-beneficial homozygote AA do. The information found in these studies indicates that ESR could be utilized as DNA marker for improvement of reproduction trait in practice of pig breeding.

In still another embodiment, the genetically modified porcine may comprise an edited or modified chromosomal sequence encoding ESR for increased litter production.

The IGF2-intron3-G3072A substitution has been recently described as the causal factor of the imprinted QTL for fat deposition and muscle growth detected within the porcine insulin-like growth factor 2 (IGF2) region. For example, studies have investigated the IGF2 substitution effect in a Large White outbred population and in an Iberian x Landrace F₂ cross. The results showed that the substitution has significant effects on fatness, growth, and shape traits with estimated effects in the expected direction.

These results agree with those obtained in the F₂ cross, where the IF2-intron3-G3072A substitution is segregating only in a small family. In addition, a QTL scan has been performed in the F₂ population for the traits used in the IGF2 substitution effect validation. Results of these studies demonstrated that there are QTL segregating in swine chromosome 2 other than the IGF2 substitution for carcass weight, LM area, and pH measured at 24 h after slaughter. The results confirm the relevance of the IGF2 substitution previously described in the literature, but one skilled in the art will also recognized there are additional valuable mutations to be revealed in this chromosome related to IGF2.

The GHRH (growth hormone releasing hormone) gene takes part in growth metabolism according to interaction with various interdependent genes, such as GH (growth hormone), IGF1 (insulin-like growth factor 1), PIT1 (pituitary-specific transcription factor 1), GHRHR (growth hormone releasing hormone receptor) and GHR (growth hormone receptor). This gene is also known to regulate the release of GH. It is located in SSC 17, and is known to be associated with back fat thickness and average daily gain due to Alul RFLP polymorphism.

The H-FABP gene is a member of the fatty acid-binding protein (FABP) family that comprises a group of small cytosolic proteins that specifically bind and intracellularly transport fatty acids and other hydrophobic ligands. In addition, FABP may regulate lipid metabolism and other cellular processes such as gene transcription, cellular signaling, growth and differentiation. For the H-FABP gene, Mspl, Haelll and Hinfl RFLP polymorphisms are known and of these, Mspl polymorphism is known to have high associations with IMF (intramuscular fat) content in pigs.

Therefore, in yet another embodiment, the genetically modified porcine may comprise an edited chromosomal sequence encoding IGF2, GHRH, H-FABP, GH, IGF1, PIT1, GHRHR, GHR or combinations thereof. The edited chromosomal sequence may comprise at least one modification such that an altered version of IGF2, GHRH, H-FABP, GH, IGF1, PIT1, GHRHR, GHR is produced. The chromosomal sequence may be modified to contain at least one nucleotide change such that the expressed protein comprises at least one amino acid change as detailed above. Alternatively, the edited chromosomal sequence may comprise a mutation such that the sequence is inactivated and no protein is made or a defective protein is made. As detailed above, the mutation may comprise a deletion, an insertion, or a point mutation. The genetically modified porcine comprising an edited IGF2, GHRH, H-FABP, GH, IGF1, PIT1, GHRHR, GHR chromosomal sequence may have an increased growth rate than an porcine in which said chromosomal region(s) is not edited.

It is also important for the pork industry to decrease pollution, specifically phosphate pollution. To address the problem of manure-based environmental pollution in the pork industry, the phytase transgenic pig was originally developed. The saliva of these pigs contains the enzyme phytase, which allows the pigs to digest the phosphorus in phytate, the most abundant source of phosphorus in the pig diet. Without this enzyme, phytate phosphorus passes undigested into manure to become the single most important manure pollutant of pork production. These studies have shown that salivary phytase provides essentially complete digestion of dietary phytate phosphorus, relieves the requirement for inorganic phosphate supplements, and reduces fecal phosphorus output by up to 75%. These pigs offer a unique biological approach to the management of phosphorus nutrition and environmental pollution in the pork industry. In a further embodiment, the genetically modified porcine may comprise an edited chromosomal sequence encoding phytase for reduction of phosphate pollution.

Additionally, the human or porcine disease- or trait-related gene may be modified to include a tag or reporter gene as are well-known. Reporter genes include those encoding selectable markers such as cloramphenicol acetyltransferase (CAT) and neomycin phosphotransferase (neo), and those encoding a fluorescent protein such as green fuorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

The genetically modified porcine may be heterozygous for the edited chromosomal sequence or sequences. In other embodiments, the genetically modified porcine may be homozygous for the edited chromosomal sequence or sequences.

The genetically modified porcine may be a member of any one of the numerous breeds of porcine, and or may be a further modification of existing genetically modified breeds. As used herein, the term “porcine” encompasses embryos, fetuses, newborn piglets, juveniles, and adult porcine organisms. In each of the foregoing iterations of suitable animals for the invention, the animal does not include exogenously introduced, randomly integrated transposon sequences.

### (II) Genetically Modified Porcine Cells

A further aspect of the present disclosure provides genetically modified porcine cells or cell lines comprising at least one edited chromosomal sequence. The disclosure also encompasses a lysate of said cells or cell lines. The genetically modified porcine cell (or cell line) may be derived from any of the genetically modified porcines disclosed herein. Alternatively, the chromosomal sequence may be edited in a porcine cell as detailed below.

The porcine cell may be any established cell line or a primary cell line that is not yet described. The cell line may be adherent or non-adherent, or the cell line may be grown under conditions that encourage adherent, non-adherent or organotypic growth using standard techniques known to individuals skilled in the art. The porcine cell or cell line may be derived from lung (e.g., AKD cell line), kidney (e.g., CRFK cell line), liver, thyroid, fibroblasts, epithelial cells, myoblasts, lymphoblasts, macrophages, tumor cells, and so forth. Additionally, the porcine cell or cell line may be a porcine stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

Similar to the genetically modified porcines, the genetically modified porcine cells may be heterozygous or homozygous for the edited chromosomal sequence or sequences.

(III) Zinc Finger-Mediated Genome Editing

In general, the genetically modified porcine or porcine cell, as detailed above in sections (I) and (II), respectively, is generated using a zinc finger nuclease-mediated genomic editing process. The process for editing a porcine chromosomal sequence comprises: (a) introducing into a porcine embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration, the sequence flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. The embryo used in the above described method typically is a fertilized one-cell stage embryo.

Components of the zinc finger nuclease-mediated method of genome editing are described in more detail below.

### (a) Zinc Finger Nuclease

The method comprises, in part, introducing into an porcine embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

### (i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275 (43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger DNA binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

### (ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a Fokl cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:1538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Exchange Polynucleotide

The method for editing chromosomal sequences may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may be at least about 80, 81, 82, 83, 84, 85, 86, 87, 88, 89, 90, 91, 92, 93, 94, 95, 96, 97, 98, or 99% identical a region of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 by to about 10,000 by in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 by in length. In other embodiments, the sequence in the exchange polynucleotide may be about 5500, 6000, 6500, 6000, 6500, 7000, 7500, 8000, 8500, 9000, 9500, or 10,000 by in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (c) Optional Donor Polynucleotide

The method for editing chromosomal sequences may further comprise introducing at least one donor polynucleotide comprising a sequence for integration into the embryo or cell. A donor polynucleotide comprises at least three components: the sequence to be integrated that is flanked by an upstream sequence and a downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide may be a DNA plasmid.

The donor polynucleotide comprises a sequence for integration. The sequence for integration may be a sequence endogenous to the porcine or it may be an exogenous sequence. Additionally, the sequence to be integrated may be operably linked to an appropriate control sequence or sequences. The size of the sequence to be integrated can and will vary. In general, the sequence to be integrated may range from about one nucleotide to several million nucleotides.

The donor polynucleotide also comprises upstream and downstream sequence flanking the sequence to be integrated. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 bp to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 bp to about 2000 bp, about 600 bp to about 1000 bp, or more particularly about 700 bp to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for editing a chromosomal sequence by integrating a sequence, the double stranded break introduced into the chromosomal sequence by the zinc finger nuclease is repaired, via homologous recombination with the donor polynucleotide, such that the sequence is integrated into the chromosome. The presence of a double-stranded break facilitates integration of the sequence. A donor polynucleotide may be physically integrated or, alternatively, the donor polynucleotide may be used as a template for repair of the break, resulting in the introduction of the sequence as well as all or part of the upstream and downstream sequences of the donor polynucleotide into the chromosome. Thus, the endogenous chromosomal sequence may be converted to the sequence of the donor polynucleotide.

### (d) Delivery of Nucleic Acids

To mediate zinc finger nuclease genome editing, at least one nucleic acid molecule encoding a zinc finger nuclease and, optionally, at least one exchange polynucleotide or at least one donor polynucleotide is delivered into the porcine embryo or cell. Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and an exchange (or donor) polynucleotide are introduced into an embryo or cell, the ratio of exchange (or donor) polynucleotide to nucleic acid encoding a zinc finger nuclease may range from about 1:10 to about 10:1. In various embodiments, the ratio of exchange (or donor) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one exchange (or donor) polynucleotide is introduced into an embryo or cell, the nucleic acids may be introduced simultaneously or sequentially. For example, nucleic acids encoding the zinc finger nucleases, each specific for a distinct recognition sequence, as well as the optional exchange (or donor) polynucleotides, may be introduced at the same time. Alternatively, each nucleic acid encoding a zinc finger nuclease, as well as the optional exchange (or donor) polynucleotides, may be introduced sequentially.

### (e) Culturing the Embryo Or Cell

The method for editing a chromosomal sequence using a zinc finger nuclease-mediated process further comprises culturing the embryo or cell comprising the introduced nucleic acid(s) to allow expression of the zinc finger nuclease.

An embryo may be cultured in vitro (e.g., in cell culture). Typically, the porcine embryo is cultured for a short period of time at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the porcine species. Routine optimization may be used, in all cases, to determine the best culture conditions for a particular species of embryo. In some cases, a cell line may be derived from an in vitro-cultured embryo (e.g., an embryonic stem cell line).

Preferably, the porcine embryo will be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal generally will comprise the disrupted chromosomal sequence(s) in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no exchange (or donor) polynucleotide, the zinc finger nuclease recognizes, binds, and cleaves the target sequence in the chromosomal sequence of interest. The double-stranded break introduced by the zinc finger nuclease is repaired by the error-prone non-homologous end-joining DNA repair pathway. Consequently, a deletion, insertion, or nonsense mutation may be introduced in the chromosomal sequence such that the sequence is inactivated.

In cases in which the embryo or cell comprises an expressed zinc finger nuclease as well as an exchange (or donor) polynucleotide, the zinc finger nuclease recognizes, binds, and cleaves the target sequence in the chromosome. The double-stranded break introduced by the zinc finger nuclease is repaired, via homologous recombination with the exchange (or donor) polynucleotide, such that a portion of the chromosomal sequence is converted to the sequence in the exchange polynucleotide or the sequence in the donor polynucleotide is integrated into the chromosomal sequence. As a consequence, the chromosomal sequence is modified.

The genetically modified porcines disclosed herein may be crossbred to create animals comprising more than one edited chromosomal sequence or to create animals that are homozygous for one or more edited chromosomal sequences. Those of skill in the art will appreciate that many combinations are possible. Moreover, the genetically modified porcines disclosed herein may be crossed with other porcines to combine the edited chromosomal sequence with other genetic backgrounds. By way of non-limiting example, suitable genetic backgrounds may include wild-type, natural mutations giving rise to known porcine phenotypes, targeted chromosomal integration, non-targeted integrations, etc.

### (IV) Applications

The animals and cells disclosed herein may have several applications. In one embodiment, the genetically modified porcine comprising at least one edited chromosomal sequence may exhibit a phenotype desired by humans. For example, modification of the chromosomal sequence encoding one of the MC1R alleles may result in porcine producing hair with desired coat color or pattern. In other embodiments, the porcine comprising at least one edited chromosomal sequence may be used as a model to study the genetics of coat color, coat pattern, and/or hair growth. Additionally, a porcine comprising at least one disrupted chromosomal sequence may be used as a model to study a disease or condition that affects humans or other animals. Non-limiting examples of suitable diseases or conditions include albinism, hair disorders, and baldness. Additionally, the disclosed porcine cells and lysates of said cells may be used for similar research purposes.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor or exchange polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations-+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of Myostatin/GDF8, CD163 Or Sialoadhesin In Model Organism Cells

Zinc finger nuclease (ZFN)-mediated genome editing may be tested in the cells of a model organism such as an porcine using a ZFN that binds to the chromosomal sequence of a hair color-related gene of the porcine cell such as MC1R, MSH receptor proteins, tyrosinase (TYR), tyrosinase-related protein 1 (TYRP1), agouti signaling protein (ASIP), melanophilin (MLPH). The particular coat color-related gene to be edited may be a gene having identical DNA binding sites to the DNA binding sites of the corresponding porcine homolog of the gene. Capped, polyadenylated mRNA encoding the ZFN may be produced using known molecular biology techniques, including but not limited to a technique substantially similar to the technique described in Science (2009) 325:433, which is incorporated by reference herein in its entirety. The mRNA may be transfected into porcine cells. Control cells may be injected with mRNA encoding GFP.

The frequency of ZFN-induced double strand chromosomal breaks may be determined using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells may generate a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. The relative intensity of the cleavage products compared with the parental band is a measure of the level of Cel-1 cleavage of the heteroduplex. This, in turn, reflects the frequency of ZFN-mediated cleavage of the endogenous target locus that has subsequently undergone imperfect repair by NHEJ.

The results of this experiment may demonstrate the cleavage of a selected myostatin/GDF8, CD163 or sialoadhesin gene locus in porcine cells using a ZFN.

### Example 2

### Genome Editing of HAL, RN, ESR, IGF2, GHRH, H-FABP, GH, IGF1, PIT1, GHRHR Or GHR In Model Organism Embryos

The embryos of a model organism such as a porcine may be harvested using standard procedures and injected with capped, polyadenylated mRNA encoding a ZFN similar to that described in Example 1. The porcine embryos may be at the 2-4 cell stage when microinjected. Control embryos were injected with 0.1 mM EDTA. The frequency of ZFN-induced double strand chromosomal breaks was estimated using the Cel-1 assay as described in Example 1. The cutting efficiency may be estimated using the CEI-1 assay results.

The development of the embryos following microinjection may be assessed. Embryos injected with a small volume ZFN mRNA may be compared to embryos injected with EDTA to determine the effect of the ZFN mRNA on embryo survival to the blastula stage.

What is claimed is:

1. A genetically modified porcine comprising at least one edited chromosomal sequence.

2. The genetically modified porcine of claim 1, wherein the edited chromosomal sequence is inactivated, is modified, or has an integrated sequence.

3. The genetically modified porcine of claim 1, wherein the edited chromosomal sequence encodes a protein chosen from myostatin/GDF8, CD163, Halothane, ESR, IGF2, GHRH, H-FABP, GH, IGF1, PIT1, GHRHR, GHR, Phytase and combinations thereof.

4. The genetically modified porcine of claim 3, wherein the protein is myostatin/GDF8, CD163 or sialoadhesion, and the edited chromosomal sequence comprises at least one mutation such that the sequence is inactivated and the protein is not produced.

5. The genetically modified porcine of claim 4, wherein the porcine has increased disease resistance.

6. The genetically modified porcine of claim 3, wherein the protein is selected from the group consisting of IGF2, GHRH, H-FABP, GH, IGF1, PIT1, GHRHR, GHR, and the edited chromosomal sequence comprises at least one mutation such that the sequence is modified and the expressed protein comprises at least one amino acid change.

7. The genetically modified porcine of claim 6, wherein the porcine has an increased growth rate compared with a porcine in which the chromosomal region is not edited.

8. The genetically modified porcine of claim 6, wherein the porcine comprises a phenotype of increased muscle mass.

9. The genetically modified porcine of claim 1, wherein the protein is encoded by HAL, RN or PPS, and the edited chromosomal sequence comprises at least one mutation such that the sequence is modified or inactivated.

10. The genetically modified porcine of claim 9, wherein the porcine has a less fat than a porcine in which the chromosomal region is not edited.

11. The genetically modified porcine of claim 3, wherein the protein is ESR, and the edited chromosomal sequence comprises at least one mutation such that the sequence is modified and the expressed protein comprises at least one amino acid change.

12. The genetically modified porcine of claim 11, wherein the porcine has an increased litter size compared to a porcine in which the chromosomal sequence is not edited.

13. The genetically modified porcine of claim 3, wherein the protein is Phytase, and the edited chromosomal sequence comprises at least one mutation such that the sequence is modified and the expressed protein comprises at least one amino acid change.

14. The genetically modified porcine of claim 13, wherein the porcine excretes less phosphate compared to a porcine in which the chromosomal sequence is not edited.

15. The genetically modified porcine of claim 1, wherein the porcine is heterozygous or homozygous for the edited chromosomal sequence.

16. The genetically modified porcine of claim 1, wherein the porcine is an embryo, a piglet, a juvenile, or an adult.

17. An porcine embryo, the embryo comprising at least one RNA molecule encoding a zinc finger nuclease that recognizes a chromosomal sequence and is able to cleave a site in the chromosomal sequence, and, optionally, (i) at least one donor polynucleotide comprising a sequence that is flanked by an upstream sequence and a downstream sequence, the upstream and downstream sequences having substantial sequence identity with either side of the site of cleavage or (ii) at least one exchange polynucleotide comprising a sequence that is substantially identical to a portion of the chromosomal sequence at the site of cleavage and which further comprises at least one nucleotide change.

18. The porcine embryo of claim 17, wherein the chromosomal sequence encodes a protein chosen from myostatin/GDF8, CD163, Halothane, ESR, IGF2, GHRH, H-FABP, GH, IGF1, PIT1, GHRHR, GHR, Phytase and combinations thereof.

19. A genetically modified porcine cell comprising at least one edited chromosomal sequence.

20. The genetically modified porcine cell of claim 19, wherein the edited chromosomal sequence encodes a protein chosen from myostatin/GDF8, CD163, Halothane, ESR, IGF2, GHRH, H-FABP, GH, IGF1, PIT1, GHRHR, GHR, Phytase and combinations thereof.
